(19) United States
(12) Patent Application Publication
(10) Pub. No.: US 2013/0345139 A1
(43) Pub. Date: Dec. 26, 2013

(54) COMPOSITION FOR NUTRITION PURPOSES

(71) Applicant: Gelita AG, Eberbach (DE)

(72) Inventors: Steffen OESSER, Glucksburg (DE); Annelore SCHOTT, Heidelberg (DE); Stephan HAUSMANNS, Heidelberg (DE)

(73) Assignee: Gelita AG, Eberbach (DE)

(21) Appl. No.: 14/011,924

(22) Filed: Aug. 28, 2013

Related U.S. Application Data
(63) Continuation of application No. PCT/EP2012/053413, filed on Feb. 29, 2012.

(30) Foreign Application Priority Data
Mar. 1, 2011 (DE) 10 2011 000 997.3

(51) Int. Cl. A61K 38/39; A61K 31/702; A61K 31/733

(52) U.S. Cl. CPC A61K 38/39; A61K 31/733; A61K 31/702
USPC 514/16.9

(57) ABSTRACT

The invention relates to a composition for nutritional purposes with an improved efficacy in relation to the build-up of the bone matrix, comprising about 1 to about 99% by weight of collagen hydrolysate and about 1 to about 99% by weight of one or more prebiotics, in each case based on the dry mass of the composition.

### CROSS REFERENCE TO RELATED APPLICATIONS

This patent application is a continuation of International Application Number PCT/EP2012/053413, filed Feb. 29, 2012, claiming the benefit of German Patent Application No. 10 2011 000 997.3, filed Mar. 1, 2011, which are each incorporated by reference.

The present invention relates to a composition for nutritional purposes, in particular for maintaining and/or improving the health of the bones.

The bones of vertebrates and therefore also those of the human owe their high strength and mechanical stability to the particular structure of the bone matrix, which is synthesised by the osteoblasts. The two essential components of the bone matrix are, on the one hand, a framework of cross-linked collagen, this being collagen type I especially in the case of the bone matrix. The cross-linking takes place here by means of the amino acids lysine and hydroxylysine. The second component is a hydroxyapatite (also called apatite-(CaOH)), which is incorporated into the bone matrix (mineralisation of the bones). This structure of the bones is roughly comparable with that of reinforced concrete, where the properties of the steel framework (corresponding to the collagen) and of the concrete (corresponding to the hydroxyapatite) supplement one another, also with the formation of an extremely load-bearing structure.

In contrast to many other types of tissue, the bone has a high ability to regenerate, i.e. the extracellular bone matrix is continuously built up and degraded again. When there is a disruption to this equilibrium, in other words an inadequate build-up of new bone matrix, bone atrophy occurs. This reduction in the bone density is called osteoporosis and may have various causes. Typically, osteoporosis occurs with increasing age (mostly from the beginning of the fifth decade of life), particularly frequently after the menopause in women (postmenopausal osteoporosis).

It is known that the loss of bone density in osteoporosis can be counteracted by the oral administration of collagen hydrolysate, i.e. of collagen peptides produced by enzymatic hydrolysis. EP 0 777 491 B1 discloses, for example, the use of collagen hydrolysate with an average molecular weight of 1 to 40 kDa for treating postmenopausal osteoporosis. It is to be assumed that this advantageous effect of collagen hydrolysate on the health of the bones is based on a stimulation of the collagen biosynthesis by means of the osteoblasts, in a similar way to how it was already possible to demonstrate this for chondrocytes in vitro (see S. Oesser and J. Seiffert (2003), Cell Tissue Research (311) 393-399).

The invention is based on the object of proposing a composition for nutritional purposes with an improved efficacy in relation to the build-up of the bone matrix.

This object is achieved according to the invention in that the composition comprises about 1 to about 99% by weight of collagen hydrolysate and about 1 to about 99% by weight of one or more prebiotics, in each case based on the dry mass of the composition.

The combination according to the invention of collagen hydrolysate with one or more prebiotics is based on the consideration here that not only does the biosynthesis of collagen have to be enabled for an effective regeneration of the bone matrix, but so does the formation and incorporation of hydroxyapatite in an adequate quantity. The limiting factor here is the supply of bone matrix with calcium, the problem not being the adequate intake of calcium with food (this is generally ensured with balanced nutrition), but the adequate resorption of the calcium received in the intestine, which is limited, in particular, because of the formation of poorly soluble calcium salts.

It has now been shown that the resorption of calcium and therefore the mineralisation of the bones can be improved by administering prebiotics. Prebiotics are generally non-digestible food constituents, which, in a targeted manner, stimulate the growth and/or the activity of specific microorganisms of the intestinal flora and thereby have a positive influence on health. The precise mechanism by which this effect of the prebiotics acts advantageously on the calcium resorption has not yet been fully explained but the aforementioned positive effect was proven, for example, for the administration of fructans to children of about 12 years of age (see S. Abrams et al. (2005), American Journal of Clinical Nutrition (82) 471-476).

The joint administration of collagen hydrolysate and prebiotics to stimulate the formation of the two essential components of the bone matrix and to thereby achieve a synergistic effect in relation to the health of the bones has not yet been described or taken into consideration in the prior art, in particular not for the purpose of preventing and/or treating osteoporosis.

The benefit of the composition according to the invention is, however, not restricted here to this specific effect, as both prebiotics and also collagen hydrolysate, as is known, have further advantageous properties with regard to health aspects. In particular, the oral administration of collagen hydrolysate has an advantageous effect on the cartilage tissue and can thereby prevent degenerative joint diseases (see A. Bello and S. Oesser (2006), Current Medical Research and Opinion (22) 2221-2232).

It should also be emphasised as particularly advantageous that the two constituents of the composition according to the invention are legally foodstuffs, in which no disadvantageous effects are known even in the case of an overdose. For this reason, the composition does not require any kind of legal pharmaceutical or other approval, but can be offered as a composition for nutritional purposes in the form of a food supplement or else as a constituent of food.

The composition may comprise according to the invention about 1 to about 99% by weight of collagen hydrolysate and about 1 to about 99% by weight of prebiotic(s), in which the sum of collagen hydrolysate and prebiotics should make up the essential proportion of the composition (in other words more than 50% by weight). In order to ensure an adequate supply of the two constituents, it is preferred if the proportion of the collagen hydrolysate and of the prebiotic(s) is in each case about 20 to about 80% by weight, more preferably in each case about 40 to about 60% by weight, in each case based on the dry mass of the composition. The composition may, in particular, comprise a mixture of the two constituents in the weight ratio of about 1:1.

The collagen hydrolysate, which is used in the scope of the present invention, is preferably produced by enzymatic hydrolysis of collagen from animal connective tissue, in particular from pigs, cattle, poultry or fish. Pig skins or hide split are frequently used as preferred starting materials. The collagen hydrolysate may, however, also be obtained by hydrolysis of collagen from the bones of various types of animals, in particular from ossein. Methods for producing corresponding hydrolysates are known from the prior art.

Advantageously, the collagen hydrolysate has an average molecular weight in the range from about 600 to about 18,000 Da. The molecular weight distribution of collagen hydrolysates can, in particular, be determined by gel permeation chromatography using defined collagen peptides as calibration peptides. The average molecular weight is preferably in the range from about 1,500 to about 8,000 Da, more preferably in the range from about 2,500 to 3,500 Da. In particular, extensive data on the stimulation of the biosynthesis of extracellular matrix proteins exist for hydrolysates in this molecular weight range.

The prebiotic(s) used in the composition according to the invention are preferably selected from oligosaccharides and/or polysaccharides. Oligosaccharides and polysaccharides make up the majority of the known prebiotically effective substances, the additional advantageous effect being produced by the use of substances of this type that the taste of the composition according to the invention is very significantly improved compared to pure collagen hydrolysate. Although collagen hydrolysates can be produced by means of corresponding methods, which are generally called taste-neutral, many consumers perceive a taste impairment designated glutinous. Surprisingly, the negative taste components of collagen hydrolysate can be almost completely eliminated by the combination with prebiotic oligosaccharides and/or polysaccharides.

The prebiotic(s) are preferably selected from inulin, fructans, galacto-oligosaccharides (GOS), fructo-oligosaccharides (FOS), resistant maltodextrins, polydextrose and mixtures thereof. These comprise both naturally occurring and synthetically produced saccharides. Inulin is a fructan, which comprises up to 100 fructose units as well as a terminal glucose unit. Fructo-oligosaccharides and galacto-oligosaccharides exclusively comprise fructose or galactose units (generally up to 10), and polydextrose is a synthetic polysaccharide made of glucose, sorbite and citric acid units.

In a preferred embodiment of the invention, the composition comprises at least one soluble calcium salt as a further constituent. This simultaneously ensures an adequate supply of the user with calcium regardless of his/her other nutritional habits.

In principle all soluble calcium salts which are not toxic or do not have other disadvantageous effects are possible. The at least one soluble calcium salt is preferably selected from calcium citrate, calcium lactate, calcium gluconate, calcium lactate gluconate, calcium lactobionate and mixtures thereof.

The quantity of the at least one calcium salt is advantageously selected in such a way that the proportion of calcium is about 0.1 to about 10% by weight, based on the dry mass of the composition. The proportion of calcium is more preferably about 0.5 to about 5% by weight.

The composition according to the invention can moreover contain further constituents that promote the health of the bones (in particular vitamin D and/or vitamin K) or that in general have an advantageous effect in terms of health (for example further minerals such as magnesium, vitamin C and/or glucosamine).

Furthermore, the composition according to the invention may contain further additives or auxiliary substances in secondary quantity proportions, such as, for example, sweeteners, flavourings, dyes and/or release agents.

According to a preferred embodiment of the invention, the composition is in the form of a powder or granulate. In this form, the composition can be stored well and measured out as desired by the user and, for example, dissolved in water or a drink. The collagen hydrolysate and the prebiotic(s) and optionally further constituents can be ground together after mixing in order to obtain a composition with a uniform particle size.

The composition according to the invention may, however, also be present in the form of a solution or a suspension (depending on the solubility of the prebiotic(s)). The provision of the composition in a ready-to-drink, liquid form of this type (for example in correspondingly sized ampoules) makes it particularly simple for the consumer to use.

The composition according to the invention for nutritional purposes has a general health-promoting effect because of its constituents. An important aspect of the present invention relates, however, to the maintaining and/or improvement of the health of the bones, in particular the prevention and/or treatment of osteoporosis, by the above-described interaction of collagen hydrolysate (stimulation of collagen synthesis) and the prebiotic(s) (improvement of the calcium resorption and thus stimulation of the storage of hydroxyapatite in the bone matrix).

The composition according to the invention is particularly suitable for preventing and/or treating postmenopausal osteoporosis. Furthermore, the administration of the composition according to the invention is also advantageous in patients with a diagnosed formation of bone metastases.

The composition according to the invention is provided for nutritional purposes in general, which, in the scope of the invention comprises every form of administration to the human or also to animals, whether as food, food supplements or a pharmaceutical preparation.

According to a preferred embodiment of the invention, the composition is used as a food supplement, in particular in the form of tablets, capsules, capsettes or sachets. Alternatively, a solution or suspension can also be offered as a food supplement (see above). This allows the user to take in a predetermined or definable quantity of the composition according to the invention in addition to his/her normal nutrition.

The daily dose, which can be divided over one or more tablets etc., preferably contains about 3 to about 15 g of the composition according to the invention, more preferably about 5 to about 10 g.

In the scope of the present invention, the composition may also, however, be used as a constituent of food (so-called “functional food”), in particular as a constituent of milk products (for example yoghurt) or confectionery (for example baked goods, chocolate bars etc.). In the course of production, either the composition according to the invention as such can be added to the food, or its individual constituents, in other words collagen hydrolysate and prebiotic, can be added thereto, optionally also at different times in the production method.

These and further advantages of the invention will be described in more detail with the aid of the following examples with reference to the Figures, in which, in detail:

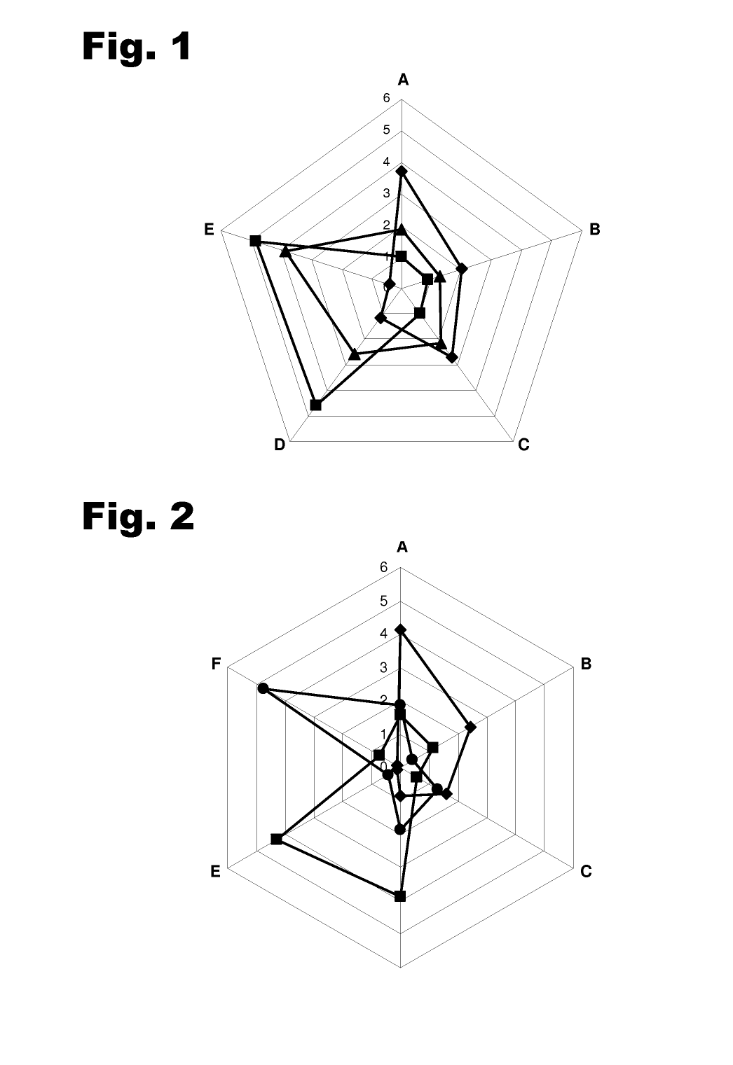

FIG. 1 shows a chart for the sensory evaluation of a composition according to the invention in comparison to its individual constituents; and

FIG. 2 shows a chart for the sensory evaluation of various compositions according to the invention.

### EXAMPLE 1

A composition according to the invention for nutritional purposes was produced in that collagen hydrolysate and a prebiotic were mixed in the weight ratio 1:1.

The product GELITA® Sol of the applicant was used as the collagen hydrolysate, which is an enzymatically hydrolysed collagen from pig skin with an average molecular weight of about 3,000 Da.

Inulin was used as the prebiotic polysaccharide, which is marketed under the name Orafti® Synergy1 by the company BENEO-Orafti.

The composition according to the invention and, as a comparison, the two individual constituents, were each dissolved in a quantity of 60 g/l in water and subjected to a sensory assessment by seven test subjects. In this case, the test subjects each undertook an assessment of the following five taste components on a scale from 0 (not perceivable) to 6 (highly pronounced):
A: glutinous
B: bitter
C: sour
D: sweet
E: caramelly

The mean values were formed in each case from the individual evaluations of the seven test subjects and shown in a spider chart with five axes arranged in a star shape (for the taste components A to E). This spider chart is shown in FIG. 1, the values for the composition according to the invention (collagen hydrolysate with inulin) being shown as squares, the values for the collagen hydrolysate being shown as rhombuses and the values for the inulin being shown as triangles.

It is clearly shown that the taste components typically perceived as unpleasant (glutinous, bitter and sour) dominate in the collagen hydrolysate. In comparison, the taste of the inulin is substantially assessed as sweet and caramelly.

Surprisingly, the taste impression of the composition according to the invention was on average assessed to be still more positive than the inulin alone, i.e. as less glutinous, bitter and sour and more caramelly and substantially sweeter. The unpleasant taste of the collagen hydrolysate is thus not only moderated by the mixture of inulin, but, on the contrary, reversed.

Owing to this taste effect, the acceptability of the composition according to the invention for the user can be significantly improved compared to pure collagen hydrolysate, which is a further important advantage in addition to the synergistic effect with regard to the health of the bones.

### EXAMPLE 2

A further composition according to the invention was produced with a different prebiotic, in that collagen hydrolysate (GELITA® Sol) was mixed in the weight ratio 1:1 with a product containing a high proportion of galacto-oligosaccharides (VIVINAL®, marketed by the company Friesland Campina Domo).

A sensory assessment was in turn carried out by seven test subjects, specifically for the composition described in Example 1 (collagen hydrolysate with inulin), the mixture of collagen hydrolysate and galacto-oligosaccharides as well as collagen hydrolysate alone.

The taste components A to E according to Example 1 were assessed as well as, additionally, the taste component “milky”, in turn on a scale from 0 to 6.

The results are shown in the spider chart shown in FIG. 2. The symbols for collagen hydrolysate (rhombuses) and collagen hydrolysate with inulin (squares) correspond to FIG. 1 and the values for collagen hydrolysate with galacto-oligosaccharides are shown by circles.

In the case of this second composition according to the invention, a significant shifting of the taste impression is shown compared to the collagen hydrolysate, in particular the taste components “glutinous” and “bitter” are significantly reduced. In contrast to the first composition, the mixture of collagen hydrolysate and galacto-oligosaccharides is described substantially as “milky”, this also, however, being a taste that is perceived as pleasant.

1. A composition for nutritional purposes, comprising about 1 to about 99% by weight of collagen hydrolysate and about 1 to about 99% by weight of one or more prebiotics, in each case based on the dry mass of the composition.

2. The composition according to claim 1, wherein the proportion of the collagen hydrolysate and of the prebiotic(s) is in each case about 20 to about 80% by weight, in each case based on the dry mass of the composition.

3. The composition according to claim 1, wherein the collagen hydrolysate is produced by enzymatic hydrolysis of collagen from animal connective tissue or bone.

4. The composition according to claim 1, wherein the collagen hydrolysate has an average molecular weight in the range from about 600 to about 18,000 Da.

5. The composition according to claim 1, wherein the prebiotic(s) are selected from oligosaccharides and/or polysaccharides.

6. The composition according to claim 1, further comprising at least one soluble calcium salt.

7. The composition according to claim 6, wherein the at least one soluble calcium salt is selected from calcium citrate, calcium lactate, calcium gluconate, calcium lactate gluconate, calcium lactobionate and mixtures thereof.

8. The composition according to claim 6, wherein the proportion of calcium is about 0.1 to about 10% by weight, based on the dry mass of the composition.

9. The composition according to claim 1, further comprising vitamin D, vitamin K, vitamin C, minerals and/or glucosamine.

10. The composition according to claim 1, wherein the composition is in the form of a powder or granulate.

11. The composition according to claim 1, wherein the composition is in the form of a solution or a suspension.

12. A method for maintaining and/or improving the heath of the bones in a patient comprising administering to the patient the composition of claim 1.

13. The method of claim 12, wherein the composition is administered to the patient as a food supplement in the form of tablets, capsules, capsettes or sachets.

14. The method according to claim 13, wherein a daily dose of about 3 to about 15 g of the composition is administered to the patient.

15. The composition according to claim 1 as a constituent of food.

16. The composition according to claim 2, wherein the proportion of the collagen hydrolysate and of the prebiotic(s) is in each case about 40 to about 60% by weight, in each case based on the dry mass of the composition.

17. The composition according to claim 4, wherein the collagen hydrolysate has an average molecular weight in the range from about 2,500 to about 3,500 Da.

18. The composition according to claim 1, wherein the prebiotic(s) are selected from inulin, fructans, galacto-oligosaccharides (GOS), fructo-oligosaccharides (FOS), resistant maltodextrins, polydextrose and mixtures thereof.

19. A method for preventing and/or treating osteoporosis in a patient, comprising administering to the patient the composition of claim 1.

20. The method according to claim 14, wherein a daily dose of about 5 to about 10 g of the composition is administered to the patient.
